(19) United States
(12) Patent Application Publication
(10) Pub. No.: US 2025/0057757 A1
(43) Pub. Date: Feb. 20, 2025

(54) Compositions for providing parenteral nutrition to pediatric patients

(71) Applicant: Fresenius Kabi Deutschland GmbH, Bad Homburg (DE)

(72) Inventors: Heidi Schuster, Bad Homburg (DE); Therese Jonsson, Bad Homburg (DE)

(21) Appl. No.: 18/721,707

(22) PCT Filed: Dec. 19, 2022
(86) PCT No.: PCT/EP2022/086604

§ 371 Date: Jun. 19, 2024

(30) Foreign Application Priority Data
Dec. 21, 2021 (EP) 21216240.8

(51) Int. Cl. A61K 9/00; A61K 9/107; A61K 31/198; A61K 31/20; A61K 31/7004

(52) U.S. Cl. CPC A61K 9/0029; A61K 9/107; A61K 31/198; A61K 31/20; A61K 31/7004

(57) ABSTRACT

The invention relates to a composition for use in providing parenteral nutrition to a pediatric patient comprised in a 3-chamber bag.

### FIELD OF THE INVENTION

The invention relates to compositions for use in providing parenteral nutrition to pediatric patients.

### BACKGROUND OF THE INVENTION

Parenteral nutrition is an essential component in the treatment of pediatric patients, where oral or enteral feeding is not sufficient to meet nutritional needs or for other reasons impossible, e.g., in preterm infants, in pediatric patients with short bowel syndrome, or in pediatric patients undergoing abdominal surgery, chemotherapy or bone marrow transplantation.

Standard compositions for providing parenteral nutrition to adults comprising amino acid solutions, glucose solutions and lipid emulsions, comprised in 3-chamber bags, are commercially widely available. However, this is not the case for children whose nutritional needs clearly differ from the nutritional needs of adults. Also, the nutritional needs of children vary according to weight and age. That is why parenteral nutrition for pediatric patients is often prescribed and compounded individually. This practice has disadvantages, e.g., there is a risk of individual mistakes in the calculation of nutritional needs, compounding errors, or ignorance of certain chemical incompatibilities within the components that need to be administered.

Hence, there is a need for compositions for use in providing parenteral nutrition to pediatric patients comprising a glucose solution, an amino acid solution and a lipid emulsion conveniently provided in ready-to-use 3 chamber bags.

### DESCRIPTION OF THE INVENTION

The present invention relates to a composition for use in providing parenteral nutrition to a pediatric patient, wherein the composition is comprised in a 3-chamber bag and comprises
(i) in the first chamber a glucose solution providing 14-22 g glucose per 100 ml of the glucose solution,
(ii) in the second chamber an amino acid solution providing 6.3-6.7 g of amino acids per 100 ml of the amino acid solution and comprising 8-10 wt. % L-Alanine, 6-9 wt. % L-Arginine, 0.5-2 wt. % L-Cysteine and/or L-Cystine, 3-5 wt. % Glycine, 3-5 wt. % L-Histidine, 4-8 wt. % L-Isoleucine, 10-13 wt. % L-Leucine, 8-11 wt. % L-Lysine, 2-4 wt. % L-Methionine, 3-5 wt. % L-Phenylalanine, 3-10 wt. % L-Proline, 4-8 wt. % L-Serine, 0.3-0.7 wt. % Taurine, 3-6 wt. % L-Threonine, 1.8-2.2 wt. % L-Tryptophan, 0.4-4.2 wt. % L-Tyrosine, and 5-9 wt. % L-Valine, based on the total weight of the amino acids, and
(iii) in the third chamber a lipid emulsion providing 20-22 g of lipids per 100 ml of the lipid emulsion and comprising 14-19 wt. % caprylic acid, 10-14 wt. % capric acid, 24-29 wt. % oleic acid, 16-21 wt. % linoleic acid, 1.5-3.5 wt. % acid eicosapentaenoic and 2-3 wt. % docosahexaenoic acid based on the total weight of the lipids, wherein the fatty acids are present in triglyceride-bound form,
   wherein the contents of the 3 chambers is mixed before the composition is intravenously administered to the pediatric patient,
   wherein the glucose solution, the amino acid solution and the lipid emulsion are comprised in the composition and administered to the pediatric patient at a volume ratio of 4.6-7.1:2.9-6.1:1, and
   wherein the composition is administered at a dose of 30-140 ml per kg body weight per day.

The composition according to the present invention is administered parenterally, preferably intravenously, after the contents of the 3 chambers has been mixed. The mixture, which is administered parenterally, preferably intravenously, has an osmolarity of not more than 1000 mOsmol/kg, preferably not more than 950 mOsmol/kg, more preferably not more than 920 mOsmol/kg. Preferably, the osmolarity of the mixture is between 800 and 1000 mOsmol/kg, more preferably between 800 and 950 mOsmol/kg, most preferably between 820 and 920 mOsmol/kg e.g., between 830 and 850 mOsmol/kg, between 880 and 900 mOsmol/kg, between 850 and 870 mOsmol/kg, or between 800 and 920 mOsmol/kg.

The pH of the mixture is between 5 and 8, preferably between 5.5 and 7.5, more preferably between 6.0 and 7.4.

### The Lipid Emulsion

The compositions according to the present invention comprise a lipid emulsion. The lipid emulsion is an oil-in-water emulsion and comprises 20 to 22 wt. % of an oil phase based on the total weight of the emulsion.

### The Oil Phase

The oil phase comprises 14-19 wt. % caprylic acid, 10-14 wt. % capric acid, 24-29 wt. % oleic acid, 16-21 wt. % linoleic acid, 1.5-3.5 wt. % eicosapentaenoic acid and 2-3 wt. % docosahexaenoic acid based on the total weight of the lipids, wherein the fatty acids are present in triglyceride-bound form.

In this context it is to be understood that the oil phase (and the lipid emulsion) may comprise minor amounts of non-esterified fatty acids, the minor amounts being within the compendial limits. Preferably, in the context of the present invention, the lipid emulsion comprised in the compositions according to the present invention, the total amount of non-esterified fatty acids does not exceed 2.8 g, more preferably 2.2 g, per liter of the lipid emulsion.

Preferably, the oil phase comprises soybean oil, olive oil, fish oil, and medium chain triglycerides.

More preferably, the oil phase comprises 30 wt. % soybean oil, 30 wt. % medium-chain triglycerides, 25 wt. % olive oil and 15 wt. % fish oil based on the total weight of the oil phase.

The term “fish oil” refers to “purified fish oil” and to “purified fish oil rich in omega 3 fatty acids”, the latter according to the European Pharmacopoeia 6.0 comprising at least 9% (w/w) of the omega-3-fatty acid docosahexaenoic acid (DHA) and at least 13% (w/w) of the omega-3 fatty acid eicosapentaenoic acid (EPA) expressed as triglycerides. Fish oils are commercially available.

In the context of the present disclosure the term “fish oil” also refers to fish oil extracts that may be further enriched or downgraded respectively in certain fatty acids. Such fish oil extracts are commercially available, e.g., from Solutex S.L.

The term “medium chain triglycerides” (MCT) refers to triglycerides of fatty acids having 6 to 12 carbon atoms in length, including caproic acid, caprylic acid, capric acid and lauric acid. MCT are commercially available.

### The Droplet Size

As the lipid emulsion comprised in the compositions according to the present invention is oil-in-water emulsion, the continuous phase is aqueous and comprises oil droplets. These oil droplets are stabilized within the aqueous phase by at least one emulsifier and optionally further additives. The size of the oil droplets depends on the qualitative and quantitative composition of the emulsion and its preparation.

The oil droplets of the emulsion preferably have a mean diameter (volume based) of 130 to 450 nm, preferably 150 to 400 nm, more preferably 180 to 350 nm, when measured directly upon sterilization using a Mastersizer 3000 (Malvern) according to USP <729>.

### The PFAT₅ Value

According to the USP in an oil-in-water emulsion for parenteral administration the percentage of fat residing in oil droplets larger than 5 μm in diameter (PFAT₅ value) must not exceed 0.05%.

Where an emulsion for parenteral administration is mixed with an amino acid solution and/or a glucose solution before administration, the PFAT₅ value should remain below 0.05% for at least 24 hours, preferably for at least 48 hours after the emulsion has been mixed with the amino acid solution and/or the glucose solution.

The PFAT₅ value is measured according to one of the methods according to USP<729>.

The lipid emulsion comprised in the compositions according to the present invention has a PFAT₅ value below 0.05%, preferably below 0.04%, more preferably below 0.3%. The PFAT₅ value remains below 0.05%, preferably below 0.04%, more preferably below 0.03%, during the shelf life of the emulsion. The shelf life of the emulsion is preferably at least 1 year, more preferably at least 1.5 years, more preferably at least 2 years, when stored at 5° C. to 25° C. at a relative humidity of 40 to 60%.

The PFAT₅ value of the lipid emulsion comprised in the compositions according to the present invention remains below 0.05% for at least 24 hours, preferably for at least 48 hours, after it has been mixed with the amino acid solution and/or the glucose solution comprised in the composition according to the present invention.

### The Emulsifier

The lipid emulsion comprised in the compositions according to the present invention comprises at least one pharmaceutically acceptable emulsifier. The term “emulsifier” refers to compounds which stabilize the composition by reducing the interfacial tension between the oil phase and the water phase and which typically comprise at least one hydrophobic group and at least one hydrophilic group. These emulsifiers (which may also be referred to as surfactants) are preferably used in amounts effective to provide, optionally together with further surfactants present, a stable and even distribution of the oil phase within the aqueous phase.

The at least one emulsifier comprises at least one phospholipid. Within the meaning of the present disclosure the term “phospholipid” refers to naturally occurring or synthetic phospholipids that may be suitably refined. Suitable phospholipids include, but are not limited to, phospholipids derived from corn, soybean, egg or other animal origin, or mixtures thereof. Phospholipids typically comprise mixtures of diglycerides of fatty acids linked to the choline ester of phosphoric acid and can contain differing amounts of other compounds depending on the method of isolation. Typically, commercial phospholipids are a mixture of acetone-insoluble phosphatides. Preferably, the phospholipids are obtained from egg or other animal origin, or from seeds including soybean and corn, using methods well known in the art. Phospholipids obtained from soybean are referred to herein as soy phospholipids. Phospholipids obtained from egg are referred to herein as egg phospholipids.

The lipid emulsion comprised in the compositions according to the present invention comprises phospholipids as emulsifier, more preferably the phospholipids are selected from the group consisting of egg phospholipids, soy phospholipids, and mixtures thereof, most preferably the phospholipids are egg phospholipids.

Such emulsifiers are commercially available.

Preferably, the emulsifier is used in an amount of 0.5 to 5% (w/v), more preferably 0.5 to 3% (w/v), most preferably 1.0 to 2.0% (w/v) based on the total volume of the emulsion.

### The Co-Surfactant

The lipid emulsion comprised in the compositions according to the present invention may further comprise a pharmaceutically acceptable co-surfactant. A co-surfactant is an amphiphilic molecule, i.e., a molecule that contains both hydrophilic and lipophilic groups. Usually, a co-surfactant substantially accumulates with the emulsifier at the interfacial layer. The hydrophile-lipophile balance (HLB) number is used as a measure of the ratio of hydrophilic and lipophilic groups present in a surfactant or co-surfactant, respectively. Preferably, a co-surfactant with a very low HLB value (thus with a relatively high affinity to oil) is used together with an emulsifier with a high HLB to modify the overall HLB of the system. Unlike the emulsifier, the co-surfactant may not be capable of forming self-associated structures, like micelles, on its own. Several kinds of molecules including nonionic emulsifiers, alcohols, amines, and acids, can function as co-surfactants in a given system. The co-surfactant is usually used in a lower amount than that of the emulsifier. Apart from modifying the overall HLB value of the system, the co-surfactant has the effect of further reducing the interfacial tension and increasing the fluidity of the interface. Co-surfactants may also adjust the curvature of the interfacial film by partitioning between the tails of the emulsifier chains, allowing greater penetration of the oil between the emulsifier tails.

Preferably, the co-surfactant is a free long chain fatty acid or a salt thereof, preferably a free unsaturated fatty acid or a salt thereof, preferably an omega-9 fatty acid or a salt thereof, more preferably a monounsaturated omega-9 fatty acid or a salt thereof, more preferably oleic acid or sodium oleate.

The total amount of the co-surfactant is preferably in the range of from 0.01% to 1%, more preferably in the range of from 0.02% to 0.5%, more preferably in the range of from 0.02% to 0.2% based on the total volume of the emulsion (w/v).

### The Tonicity Agent

The lipid emulsion comprised in the compositions according to the present invention may comprise at least one pharmaceutically acceptable tonicity agent. Tonicity agents are used to confer tonicity. Suitable tonicity agents may be selected from the group consisting of sodium chloride, mannitol, lactose, dextrose, sorbitol, glycerol, and mixtures thereof. Preferably, the tonicity agent is glycerol.

Preferably, the total amount of tonicity agents is in the range of 0.1 to 10%, more preferably from 1% to 5%, more preferably from 1% to 4%, more preferably 1% to 3%, more preferably from 1.5% to 2.8%, and even more preferably from 2.0% to 2.5% based on the total volume of the emulsion (w/v).

In case the tonicity agent is glycerol the preferred amount is 2.0% to 2.8%, the most preferred amount is 2.1% to 2.6% based on the total volume of the emulsion (w/v).

Preferably, the lipid emulsion has an osmolality in the range of 200 to 400 mOsmol/kg, more preferably between 250 and 350 mOsmol/kg, most preferably between 250 and 300 mOsmol/kg.

### The Antioxidant

The lipid emulsion comprised in the compositions according to the present invention may comprise at least one pharmaceutically acceptable antioxidant.

An antioxidant may be any pharmaceutically acceptable compound having antioxidant activity, for example, the antioxidant may be selected from the group consisting of sodium metasulfite, sodium bisulfite, sodium sulfite, sodium thiosulfate, thioglycerol, thiosorbitol, thioglycolic acid, cysteine hydrochloride, n-acetyl-cysteine, citric acid, alpha-tocopherol, beta-tocopherol, gamma-tocopherol, delta-tocopherol, tocotrienols, soluble forms of vitamin E, butylated hydroxyanisole (BHA), butylated hydroxytoluene (BHT), t-butylhydroquinone (TBHQ), monothioglycerol, propyl gallate, histidine, enzymes such as superoxide dismutase, catalase, selenium glutathione peroxidase, phospholipid hydroperoxide and glutathione peroxidase, Coenzyme Q10, carotenoids, quinones, bioflavonoids, polyphenols, bilirubin, ascorbic acid, isoascorbic acid, uric acid, metal-binding proteins, ascorbic acid palmitate, and mixtures thereof. The at least one antioxidant is particularly selected from the group consisting of alpha tocopherol, beta tocopherol, gamma tocopherol, delta tocopherol, tocotrienols, ascorbic acid, and mixtures of two or more thereof. Preferably, the antioxidant is alpha tocopherol or mixture of alpha-, beta- and gamma-tocopherol.

If present, the total amount of agents with antioxidant activity is preferably in the range of from 0.01% to 0.05%, more preferably from 0.01% to 0.04%, more preferably from 0.01% to 0.03%, and even more preferably from 0.015% to 0.025% based on the total volume of the emulsion (w/v).

### The pH Adjusting Agent

The pH of the lipid emulsion comprised in the compositions according to the present invention may be adjusted by adding solutions of conventionally known acids or bases such as HCl and NaOH or by using buffers, such as phosphate buffers.

The final pH of the emulsion is preferably in the range of from 7.0 to 10.0, more preferably between 7.5 and 9.5, most preferably between 7.5 and 9.0.

Preferably, the pH of the oil-in-water emulsions manufactured according to the process of the present invention is adjusted using a solution of NaOH.

### The Preservative

The lipid emulsion comprised in the compositions according to the present invention may further comprise a pharmaceutically acceptable preservative.

Suitable preservatives are 4-hydroxybenzoic acid as well as salts and esters thereof, sorbic acid as well as salts and derivatives thereof, thiomersal, chlorbutanol, chlorhexidine and salts thereof, phenylmercury salts, p-chlorocresol, ethylenediamine-tetraacetic acid and salts thereof, phenoxyethanol or mixtures thereof.

Typically, the preservative is used in concentrations between 0.001 and 2.0 wt. % based on the total weight of the emulsion.

Preferably, the preservative is ethylenediaminetetraacetic acid or a pharmaceutically acceptable salt thereof.

Where the preservative is ethylenediaminetetraacetic acid or a pharmaceutically acceptable salt thereof, it is preferably used in a concentration of 0.05 to 0.8 wt. %, preferably 0.1 to 0.7 wt. %, based on the total weight of the emulsion.

### Preparation of the Lipid Emulsion

The lipid emulsion comprised in the compositions according to the present invention is manufactured by a process comprising the following steps:
(a) providing an oil phase comprising 14-19wt. % caprylic acid, 10-14 wt. % capric acid, 24-29 wt. % oleic acid, 16-21 wt. % linoleic acid, 1.5-3.5 wt. % eicosapentaenoic acid and 2-3 wt. % docosahexaenoic acid based on the total weight of the lipids, wherein the fatty acids are present in triglyceride-bound form,
(b) providing an aqueous phase 1 comprising water,
(c) obtaining a pre-emulsion by mixing the oil phase provided in step a) with the aqueous phase 1 provided in step b),
(d) obtaining a first emulsion by homogenizing the pre-emulsion obtained in step c),
(e) providing an aqueous phase 2 comprising water,
(f) obtaining the oil-in-water emulsion by mixing the first emulsion obtained in step d) with the aqueous phase 2 provided in step e) and
(g) sterilizing the oil-in-water emulsion obtained in step f) and filling it into a suitable container either before or after sterilization.

### Step A—Providing the Oil Phase

Step a) is preferably carried out by mixing the oil or oils and optionally a pharmaceutically acceptable antioxidant and/or a pharmaceutically acceptable co-surfactant. This step is preferably carried out by mixing, e.g., by means of an Ultra-Turrax, e.g., at 5000 rpm, e.g., for 5 minutes, at a temperature of 55 to 85° C., e.g., at 60 to 70° C., until a homogeneous and clear phase is obtained. In particular, it is to be understood that the at least one pharmaceutically acceptable emulsifier may be added either in step a) or in step b).

Preferably, where the emulsifier is added in step a) the emulsifier is added after the oil phase has been heated to 55 to 85° C.

### Step B—Providing the Aqueous Phase 1

Step b) is preferably carried out by providing water for injection and optionally adding a pharmaceutically acceptable tonicity agent and/or a pharmaceutically acceptable co-surfactant and/or a pharmaceutically acceptable preservative.

Optionally, the pH of the aqueous phase 1 is adjusted to 8.5-10.0, preferably to 9.0 to 10.0.

The aqueous phase is then heated to a temperature of 55 to 85° C., e.g., to 60 to 70° C.

In particular, it is to be understood that the at least one pharmaceutically acceptable emulsifier may be added either in step a) or in step b).

Preferably, where the emulsifier is added in step b) the emulsifier is added after the aqueous phase has been heated to 55 to 85° C.

### Step C—Obtaining the Pre-Emulsion

In step c) the oil phase provided in step a) is mixed with the aqueous phase 1 provided in step b) thereby forming a pre-emulsion. The mixing may be carried out by any method known to those skilled in the art, e.g., by means of an Ultra-Turrax, e.g., for 5 to 15 minutes, e.g., for 10 to 12 minutes at e.g., 5000 to 15000 rpm, e.g., at 10000 rpm.

Preferably, the oil phase is added to the aqueous phase or vice-versa at a temperature in the range of from 55 to 85° C., e.g., at a temperature between 60 and 70° C.

Optionally, the pH of the pre-emulsion may be adjusted to a pH in the range of from 8.5 to 10.0, preferably to pH from 9.0 to 10.0.

Optionally, water for injection is added to compensate for the potential loss of water during processing the pre-emulsion.

The concentration of the oil phase in the pre-emulsion obtained in step c) and in the first emulsion obtained n step d) is higher than the concentration of the oil phase in the emulsion obtained in step f). This is because in step f) the first emulsion obtained in step d) is diluted with the aqueous phase 2 provided in step e).

Preferably, the concentration of the oil phase in steps c) and d) is at least 130% of the concentration of the oil phase in the emulsion obtained in step f), e.g., 130% to 330% of the concentration of the oil phase in the emulsion obtained in step f).

More preferably, the concentration of the oil phase in steps c) and d) is at least 150% of the concentration of the oil phase in the emulsion obtained in step f), e.g., 150% to 330% of the concentration of the oil phase in the emulsion obtained in step f).

Most preferably, the concentration of the oil phase in steps c) and d) is at least 180% of the concentration of the oil phase in the emulsion obtained in step f), e.g., 180% to 330%, 180% to 300% or 180% to 250% of the concentration of the oil phase in the emulsion obtained in step f).

In a particularly preferred embodiment, the concentration of the oil phase in steps c) and d) is 200% of the concentration of the oil phase in the emulsion obtained in step f).

### Step D—Obtaining the First Emulsion

In step d) the pre-emulsion obtained in step c) is homogenized, e.g., by means of a high-pressure homogenizer or a counter-jet disperser, preferably at a temperature of 40 to 80° C., more preferably at a temperature of 50 to 75° C., most preferably at a temperature of 60 to 70° C.

Optionally, in step d) the pH is adjusted to values between 8.5 and 10.0, preferably to values between 9.0 and 10.0.

### Step E—Providing the Aqueous Phase 2

Step e) is preferably carried out by providing water for injection and optionally adding a pharmaceutically acceptable tonicity agent and/or a pharmaceutically acceptable co-surfactant and/or a pharmaceutically acceptable preservative. Optionally, the pH of the aqueous phase 2 is adjusted to 8.5 to 10.0, preferably to 9.0 to 10.0.

### Step F—Obtaining the Emulsion

In step f) the first emulsion obtained in step d) is mixed with the appropriate amount of aqueous phase 2 provided in step e) to obtain the oil-in-water emulsion with desired concentration of oil phase being 20 to 22 wt. % based on the total weight of the emulsion.

Preferably, the first emulsion obtained in step d) is cooled to 20 to 40° C. before it is mixed with the water phase 2.

Optionally, the pH of the emulsion is adjusted to 8.5 to 10.0, preferably to 9.0 to 10.0.

### Step G—Sterilizing the Emulsion

In step g) the oil-in-water emulsion obtained in step f) is further sterilized to ensure its suitability for parenteral administration.

The sterilization may be carried out by any suitable method known to those skilled in the art.

Preferably, the sterilization is carried out by autoclaving, preferably at a temperature in the range of from 119 to 122° C., more preferably at a temperature around 121° C., preferably for 1 minute to 30 minutes, preferably for 10 to 15 minutes.

### The Glucose Solution

The compositions according to the present invention comprise a glucose solution comprising 14-22 g of glucose per 100 ml of the glucose solution.

In a preferred embodiment, the glucose solution comprises 17.5-18.4 g of glucose per 100 ml of the glucose solution. In a particularly preferred embodiment, the glucose solution comprises 18.2 g of glucose per 100 ml of the glucose solution.

In another preferred embodiment, the glucose solution comprises 19.5-19.7 g of glucose per 100 ml of the glucose solution. In another particularly preferred embodiment, the glucose solution comprises 19.6 g of glucose per 100 ml of the glucose solution.

In yet another preferred embodiment, the glucose solution comprises 21-22 g of glucose per 100 ml of the glucose solution. In yet another particularly preferred embodiment, the glucose solution comprises 21.6 g of glucose per 100 ml of the glucose solution.

Preferably, where the pediatric patient is an infant, preferably a preterm infant of one day or two days of age, the glucose solution comprises 21-22 g, preferably 21.6 g, of glucose per 100 ml of the glucose solution.

Preferably, where the pediatric patient is an infant between 3 and 28 days of age, the glucose solution comprises 19.5-19.7 g, preferably 19.6 g, of glucose per 100 ml of the glucose solution.

Preferably, where the pediatric patient is between 27 days and 18 years of age, the glucose solution comprises 17.5-18.4 g, preferably 18.2 g, of glucose per 100 ml of the glucose solution.

The glucose solution preferably has a pH of 3 to 7, e.g., 3.2 to 6.5, or 4.0 to 6.5 and pH may be adjusted, e.g., by adding a solution of NaOH.

The glucose solution has an osmolarity of 800 to 1300 mOsmol/kg, preferably 850 to 1250 mOsmol/kg, more preferably 900 to 1200 mOsmol/kg, e.g., 1200 mOsmol/kg, 1100 mOsmol/kg, or 1000 mOsmol/kg.

### The Amino Acid Solution

The compositions according to the present invention comprise an amino acid solution providing 6.3 to 6.7 g, preferably 6.4 to 6.7 g, more preferably 6.5 to 6.6 g, of amino acids per 100 ml of the amino acid solution.

The amino acid solution comprises 8-10 wt. % L-Alanine, 6-9 wt. % L-Arginine, 0.5-2 wt. % L-Cysteine and/or L-Cystine, 3-5 wt. % Glycine, 3-5 wt. % L-Histidine, 4-8wt. % L-Isoleucine, 10-13wt. % L-Leucine, 8-11 wt. % L-Lysine, 2-4 wt. % L-Methionine, 3-5 wt. % L-Phenylalanine, 3-10 wt. % L-Proline, 4-8 wt. % L-Serine, 0.3-0.7 wt. % Taurine, 3-6 wt. % L-Threonine, 1.8-2.2 wt. % L-Tryptophan, 0.4-4.2 wt. % L-Tyrosine, and 5-9 wt. % L-Valine, based on the total weight of the amino acids.

Preferably, the amino acid solution comprises 8.0-9.7 wt. % L-alanine, 6.2-8.4 wt. % L-arginine, 0.5-1.9 wt. %-Cysteine and/or L-Cystine, 3-5 wt. % Glycine, 3.2-4.8 wt. % L-Histidine, 4.7-8.0 wt. % L-Isoleucine, 10.0-13.0 wt. % L-Leucine, 8.5-11.0 wt. % L-Lysine, 1.9-3.2wt. % L-Methionine, 3.7-4.2 wt. % L-Phenylalanine, 3.0-9.7 wt. % L-Proline, 4.0-7.7 wt. % L-Serine, 0.4-0.6 wt. % Taurine, 3.7-5.5 wt. % L-Threonine, 2.0-2.2 wt. % L-Tryptophan, 0.4-4.2 wt. %

L-Tyrosine, and 5.5-9.0wt. % L-Valine, based on the total weight of the amino acids.

More preferably, the amino acid solution and comprises 9-10 wt. % L-Alanine, 5.5-7.0 wt. % L-Arginine, 1-2 wt. % L-Cysteine and/or L-Cystine, 2.5-4.0 wt. % Glycine, 2.5-4.0 wt. % L-Histidine, 4.0-5.5 wt. % L-Isoleucine, 10.0-11.5 wt. %

L-Leucine, 8-9 wt. % L-Lysine, 1.5-2.5 wt. % L-Methionine, 3.5-5.0wt. % L-Phenylalanine, 8-9 wt. % L-Proline, 5.0-6.5 wt. % L-Serine, 0.4-0.6wt. % Taurine, 5-6 wt. % L-Threonine, 1.9-2.2wt. % L-Tryptophan, 0.5-1.0wt. % L-Tyrosine, 7-8 wt. % L-Valine, 5.5-7.0 wt. % aspartic acid, and 10.0-11.7 wt. % glutamic acid, based on the total weight of the amino acids.

In a particularly preferred embodiment, the amino acid solution provides 6.5 g, preferably 6.53 g, of amino acids per 100ml of the amino acid solution and comprises 9.65 wt. % L-alanine, 6.28% L-arginine, 6.28 wt. % L-aspartic acid, 1.53 wt. % L-cysteine/L-cystine, 10.87% L-glutamic acid, 3.22 wt. % glycine, 3.22 wt. % L-histidine, 4.75 wt. % L-isoleucine, 10.72 wt. % L-leucine, 8.58 wt. % L-lysine, 1.99 wt. % L-methionine, 4.14 wt. % L-phenylalanine, 8.58 wt. % L-proline, 5.82 wt. % L-serine, 0.46 wt. % taurine, 5.51 wt. % L-threonine, 2.14 wt. % L-tryptophan, 0.77 wt. % L-tyrosine, and 5.51 wt. % L-valine, based on the total weight of the amino acids.

Preferably, where the pediatric patient is an infant, preferably a preterm infant of 1 day or 2 days of age, the amino acid solution does not further comprise electrolytes.

Preferably, the pH of the amino acid solution is between 4.5 and 6.0, more preferably between 4.8 and 5.8, most preferably 5.0 and 5.4. It may be adjusted, e.g., by adding a solution of acetic acid.

Preferably, the osmolarity of the amino acid solution is between 420 and 600 mOsmol/kg, more preferably between 480 and 580 mOsmol/kg, most preferably between 500 and 540 mOsmol/kg.

In some embodiments, the amino acid solution may further comprise electrolytes.

In certain embodiments, the amino acid solution further comprises Ca²⁺, Mg²⁺, Na⁺, K⁺, and phosphate.

In preferred embodiments, the amino acid solution comprises 20-31 mMol Ca²⁺, 3-6 mMol Mg²⁺, 51-58 mMol Na⁺, 46-53 mMol K⁺, 46-53 mMol Cl⁻, 0-16 mMol acetate, 20-27 mMol phosphate, and 0-6 mMol sulfate per liter of the amino acid solution.

Preferably, where the pediatric patient is an infant, preferably a preterm infant between 3 days and 28 days of age, the amino acid solution further comprises 30 mMol Ca²⁺, 4 mMol Mg²⁺, 52 mMol Na⁺, 47 mMol K⁺, 47 mMol Cl⁻, and 26 mMol phosphate per liter of the amino acid solution.

Preferably, where the pediatric patient is between 27 days and 18 years of age, the amino acid solution further comprises 21 mMol Ca²⁺, 5 mMol Mg²⁺, 57 mMol Na⁺, 52 mMol K⁺, 52 mMol Cl⁻, and 21 mMol phosphate per liter of the amino acid solution.

Where the amino acid solution further comprises electrolytes, the osmolarity is between 500 and 900 mOsmol/kg, preferably between 600 and 900 mOsmol/kg, more preferably between 700 and 900 mOsmol/kg, e.g., between 870 and 890 mOsmol/kg.

### Administration

The compositions according to the present invention are for use in providing parenteral nutrition. Hence, they are adapted for parenteral administration. Preferably, the compositions according to the present invention are administered intravenously, either into a peripheral or a central vein.

Compositions for parenteral administration must be sterile, pyrogen-free, well tolerated, free of particulate impurities and storage stable. Their pH should be as close as possible to the pH of the blood.

### The Dosage

The compositions according to the present invention are for use in providing parenteral nutrition to pediatric patients.

They are administered at doses of 30-140 ml per kg body weight per day, wherein the volume ratio of glucose solution to amino acid solution to lipid emulsion is 4.6-7.1:2.9-6.1:1.

Where the pediatric patient is an infant, preferably a preterm infant, the compositions are administered at a dose of 70-80 ml per kg body weight per day on day 1 after birth and at a dose of 90-100 ml per kg body weight per day on day 2 after birth, wherein the volume ratio of glucose solution to amino acid solution to lipid emulsion is 6.8-7.0:5.9-6.1:1, preferably 6.9:6.0:1.

Hence, where the pediatric patient is an infant, preferably a preterm infant of 1 day or 2 days of age, the composition according to the present invention preferably comprises in the first chamber 123-125 ml, preferably 124 ml, of the glucose solution, in the second chamber 107-109 ml, preferably 108 ml, of the amino acid solution, and in the third chamber 17.4-18.4 ml, preferably 17.9 ml, of the lipid emulsion.

Where the pediatric patient is an infant, preferably a preterm infant, the compositions are administered at a dose of 120-140 ml per kg body weight per day on day 3 to day 28 after birth, wherein the volume ratio of glucose solution to amino acid solution to lipid emulsion is 4.5-4.7:3.4-3.6:1, preferably 4.6:3.5:1.

Hence, where the pediatric patient is an infant, preferably a preterm infant, of 3 to 28 days of age, the composition according to the present invention preferably comprises in the first chamber 254-256 ml, preferably 255 ml, of the glucose solution, in the second chamber 191-19 2ml of the amino acid solution, and in the third chamber 53-54 ml of the lipid emulsion.

Where the pediatric patient is between 1 day and 27 days of age, the compositions are administered at a dose of 110-130 ml per kg body weight per day, preferably at a dose of 120 ml per kg body weight per day, wherein the volume ratio of glucose solution to amino acid solution to lipid emulsion is 5.2-5.4:2.9-3.1:1, preferably 5.3:3.0:1.

Where the pediatric patient is between 27 days and 2 years of age, the compositions are administered at a dose of 80-110 ml per kg body weight per day, preferably at a dose of 90-100 ml per kg body weight per day, wherein the volume ratio of glucose solution to amino acid solution to lipid emulsion is 5.2-5.4:2.9-3.1:1, preferably 5.3:3.0:1.

Where the pediatric patient is between 2 years and 11 years of age, the compositions are administered at a dose of 50-90 ml, preferably at a dose of 60-80 ml, per kg body weight per day, wherein the volume ratio of glucose solution to amino acid solution to lipid emulsion is 5.2-5.4:2.9-3.1:1, preferably 5.3:3.0:1.

Where the pediatric patient is between 12 years and 18 years of age, the compositions are administered at a dose of 30-60 ml, preferably at a dose of 40-50 ml, per kg body weight per day, wherein the volume ratio of glucose solution to amino acid solution to lipid emulsion is 5.2-5.4:2.9-3.1:1, preferably 5.3:3.0:1.

Hence, where the pediatric patient is between 1 day and 18 years of age, the composition according to the present invention preferably comprises in the first chamber 571-574 ml, preferably 573 ml, of the glucose solution, in the second chamber 318-320 ml of the amino acid solution, and in the third chamber 108-109 ml of the lipid emulsion, or the composition according to the present invention comprises in the first chamber 857-861 ml, preferably 859 ml, of the glucose solution, in the second chamber 477-480 ml of the amino acid solution, and in the third chamber 162-163 ml of the lipid emulsion.

It is to be understood that the dosages may further be adjusted according to certain specific needs of individual patients.

### The 3-Chamber Bag

The compositions according to the present invention are comprised in 3 chamber bags, wherein the first chamber comprises the glucose solution, the second chamber comprises the amino acid solution, and the third chamber comprises the lipid emulsion.

The 3-chamber-bags may be made of any suitable material substantially inert against the ingredients of the composition according to the invention, preferably even upon heat treatment, more preferably sterilization. Preferably, the bag material is plastic. In other words, the walls of the bag are made of a plastic material, e.g., a thermoplastic elastomer. The plastic material may preferably comprise one or more polymers and optionally further additives. In a further preferred embodiment, the container is transparent or tinted. In particular a tinted plastic bag, preferably a plastic bag with a tinted outer layer, advantageously reduces the amount of UV radiation that may reach the contents of the container. However, even a transparent container may comprise means to block and/or absorb UV radiation. In a further preferred embodiment, the plastic container material comprises 3 layers. In other words, the walls of the container comprise 3 layers of plastic material. The first layer is also referred to as the inner layer. The second layer is also referred to as the middle layer, and the third layer is also referred to as the outer layer. Preferably, the first or inner layer is in direct contact with the contents of the plastic bag. The second layer and the third layer are preferably not in direct contact with the contents of the plastic bag. Preferably, the middle layer is thicker than the inner layer and the outer layer, providing for requisite stability. In addition, it was found that the increased thickness of the middle layer provides for an enhanced protection against oxygen permeation from the outside to the inside of the bag. Preferably, the inner, the middle and the outer layer all comprise a thermoplastic elastomer (TPE), wherein preferably, the content in TPE is highest in the middle layer, warranting the required flexibility. The inner layer, in addition to the TPE, preferably comprises a polyolefine co-polymer. Preferably, the polyolefine co-polymer comprises a polypropylene-polyethylene co-polymer. Preferably, the TPE is a styrenic block co-polymer, more preferably Styrene-Ethylen-ButylenStyrene (SEBS). The inner layer preferably comprises 70 to 90 wt. % of the polyolefine co-polymer and 10 to 30 wt. % of the TPE, more preferably 80 wt. % of the polyolefine co-polymer and 20 wt. % of the TPE. Preferably, the inner layer has a thickness of 10 to 90 μm, more preferably 10 to 70 μm, more preferably 10 to 50 μm, more preferably 20 to 40 μm. Most preferably, the inner layer has a thickness of 30 μm. The middle layer, in addition to the TPE, preferably comprises a polyolefine co-polymer. Preferably, the polyolefine co-polymer comprises a polypropylene-polyethylene co-polymer. Preferably, the TPE comprises a styrenic block co-polymer, more preferably 2 styrenic block co-polymers, most preferably Styrene-Ethylen-Butylen-Styrene (SEBS) and Styrene-IsoprenStyrene (SIS). The middle layer preferably comprises 40 to 70 wt. %, more preferably 50 to 60 wt. % of the polyolefine co-polymer and 30 to 60 wt. %, more preferably 40 to 50 wt. % of the TPE. Most preferably the middle layer comprises 55 wt. % of the polyolefine co-polymer and 45 wt. % of the TPE. Preferably, the middle layer has a thickness of 30 to 200 μm, more preferably 50 to 190, even more preferably 70 to 180 μm, even more preferably 100 to 150 μm and most preferably 125 μm. The outer layer, in addition to the TPE preferably comprises a polyolefine. Preferably, the polyolefine comprises polypropylene, preferably an isotactic polypropylene. Even more preferably the polypropylene has UV absorption maxima at a wavelength of 290-300 nm, 330 nm, and 370 nm. This allows for an improved protection of the contents of the bag.

The chambers of the 3-chamber-bag are preferably separated by seals, more preferably by leak tight and/or peelable seals. The seals can be made by any means that allows for a separation of the contents of the container in their respective chambers during heat treatment, storing and/or transport of the container while allowing a rupturing (and thus mixing of the contents of the container) when the container is to be used as intended. Preferably, the seals are formed by fusion, preferably by welding, of regions of the opposing inner layers of the container. Such regions preferably have the shape of lines. Peelable seals preferably comprise rupture zones that allow for an easier rupturing of the seals at predetermined positions. The term leak tight seal is meant to refer to a seal which is suitable to reliably separate at least two chambers of a multi-chamber container during production, heat treatment and/or transport of the container. While the leak tight seals may be opened by any suitable means the term peelable seal is meant to refer to a leak tight seal which can be opened, preferably by application of external pressure to the container. More preferably the amount of pressure needed in order to open the peelable seal is low enough to easily open the seal by manually applying an external force to the container, most preferably by means of rolling up the container. The peelable seal furthermore preferably comprises a rupture zone which can also be described as a predetermined breaking point. Such rupture zones can, e.g., be generated by a stronger curvature of the seal and allow for a reliable opening of the seal upon application of external pressure to the container. Preferably, the container comprises at least two leak tight seals, more preferably three leak tight seals to separate the first, second, and third chamber. In one preferred embodiment the first and the second chamber are separated by a first leak tight seal and the second and third chamber are separated by a second leak tight seal. In a further and more preferred embodiment the first and second chamber are separated by a first leak tight seal, the second and third chamber are separated by a second leak tight seal and the first and third chamber are separated by a third leak tight seal. Even more preferred, the first and/or the, second and/or the third leak tight seal is/are a peelable seals. Preferably, the container according to the invention further comprises a suspension means, preferably in the form of an opening. The suspension means allows to hang the container and to withdraw the contents more easily and completely. Most preferably, the suspension means allows for the bedside administration of the contents of the container to a patient. The suspension means is therefore preferably located at the top of the container, more preferably at the top short edge of an essentially rectangular shaped container or bag. In a further preferred embodiment, the peelable seals of the container rupture upon application of external pressure to the container. The external pressure can be provided by any suitable means, e.g., by squeezing of the bag. Even more preferably the peelable seals of the container rupture upon rolling up the container. If the container is in the form of a bag with an essentially rectangular shape the rolling up is preferably started from a short edge of the container into the direction of the opposing second short edge of the container. Even more preferably the rolling up is started from the top of the container. In a further preferred embodiment, the peelable seals of the container more preferably rupture consecutively, most preferably if the external pressure is applied by a rolling up of the container. This allows for a sequential mixing of the components comprised by the container. The bag may optionally further be comprised in an overpouch. The overpouch may comprise several layers comprised of different materials. Preferably, the overpouch is transparent and/or impermeable to oxygen.

Each chamber comprises one port which serves as a port for filling the corresponding chamber of the bag.

The ports are welded into a weld seam of the bag. For this purpose, each port comprises a corresponding weld-in section. In one embodiment the weld-in section has an elongated, in particular ship-shaped, design. Preferably, the ports are welded into a transverse weld seam of the bag such that the ports are positioned on the bag side which is opposite to the bag side where the suspension means are located.

The weld-in section merges into a flexible, clampable area. During bag filling process, this area can be clamped off and therefore provides a valve function.

After completion of the bag filling, the clampable area can be pressed shut until an upper part is positioned onto the port (as a lower part) to close the inlet to each chamber.

The upper part is fixed to the lower part by a snap-on connection. Each upper part carries a sealing element for sealing the port and therefore the inlet to the chamber. The sealing element is positioned between the lower part and the upper part and fixed by clamping between the lower part and the upper part. One first port, preferably a lateral outer port, provides the lower part of a blind port. The blind port is used to fill the chamber only, but not to add or remove any liquid. Therefore, the blind port is closed with a cap as an upper part only. One second port, preferably the middle port, provides the lower part of a connector serving as an injection port. The connector further comprises the upper part. The upper part is provided with a break-off part, for instance provided as a cap. In one embodiment the break-off part contains an arrow pointing to the container and thereby indicating the connector as an injection port. After removal of the break-off part the upper part serves as a connector part for connecting an injection device. The injection of an active ingredient, for example, can take place by means of a needle syringe. In one embodiment the lower part additionally comprises an internal tube for guiding the needle insertion. This reduces the risk of needle piercing the wall of the lower part of the injection port.

One third port, preferably the opposite lateral outer port, provides the lower part of a connector serving as an infusion port. The connector further comprises the upper part. The upper part is provided with a break-off part, for instance provided as a cap. In one embodiment the break-off part contains an arrow pointing away from the container and thereby indicating the connector as an infusion port. After removal of the break-off part the upper part serves as a connector part for connecting an infusion device. The infusion respectively removal of liquid is generally carried out by inserting a spike as an infusion device. The spike is connected to an administration set to transfer the liquid from the bag into the patient.

The sealing elements of the injection port and of the infusion are preferably of different design. The sealing element of the injection port is a resealable sealing element adapted to be pierced by a needle in a fluid-tight fashion and adapted to reseal after the removal of the needle. The sealing element of the infusion port is a resealable sealing element adapted to be pierced by a spike in a fluid-tight fashion and adapted to reseal after the removal of the spike.

The present invention relates to 3-chamber bags comprising in the first chamber a glucose solution as described herein above, in the second chamber an amino acid solution as described herein above, and in the third chamber a lipid emulsion as described herein above.

In a preferred embodiment, the 3-chamber bag comprises in the first chamber 123 to 125 ml of a glucose solution proving 21 to 22 g glucose per 100 ml of the glucose solution, in the second chamber 107 to 109 ml of an amino acid solution providing 6.3 to 6.7 g of amino acids per 100 ml of the amino acid solution and comprising 8-10 wt. % L-Alanine, 6-9 wt. % L-Arginine, 0.5-2 wt. % L-Cysteine and/or L-Cystine, 3-5 wt. % Glycine, 3-5 wt. % L-Histidine, 4-8 wt. % L-Isoleucine, 10-13 wt. % L-Leucine, 8-11 wt. % L-Lysine, 2-4 wt. % L-Methionine, 3-5 wt. % L-Phenylalanine, 3-10 wt. % L-Proline, 4-8 wt. % L-Serine, 0.3-0.7 wt. % Taurine, 3-6 wt. % L-Threonine, 1.8-2.2 wt. % L-Tryptophan, 0.4-4.2 wt. % L-Tyrosine, and 5-9 wt. % L-Valine, based on the total weight of the amino acids or of an amino acid solution providing 6.3 to 6.7 g of amino acids per 100 ml of the amino acid solution and comprising 8.0-9.7 wt. % L-alanine, 6.2-8.4 wt. % L-arginine, 0.5-1.9 wt. %-Cysteine and/or L-Cystine, 3-5 wt. % Glycine, 3.2-4.8 wt. % L-Histidine, 4.7-8.0 wt. % L-Isoleucine, 10.0-13.0 wt. % L-Leucine, 8.5-11.0 wt. % L-Lysine, 1.9-3.2 wt. % L-Methionine, 3.7-4.2 wt. % L-Phenylalanine, 3.0-9.7 wt. % L-Proline, 4.0-7.7 wt. % L-Serine, 0.4-0.6 wt. % Taurine, 3.7-5.5 wt. % L-Threonine, 2.0-2.2 wt. % L-Tryptophan, 0.4-4.2 wt. % L-Tyrosine, and 5.5-9.0 wt. % L-Valine, based on the total weight of the amino acids, preferably of an amino acid solution providing 6.4 to 6.7 g amino acids per 100 ml of the amino acid solution and comprising 9-10 wt. % L-Alanine, 5.5-7.0 wt. % L-Arginine, 1-2 wt. % L-Cysteine and/or L-Cystine, 2.5-4.0 wt. % Glycine, 2.5-4.0 wt. % L-Histidine, 4.0-5.5 wt. % L-Isoleucine, 10.0-11.5 wt. % L-Leucine, 8-9 wt. % L-Lysine, 1.5-2.5 wt. % L-Methionine, 3.5-5.0 wt. % L-Phenylalanine, 8-9 wt. % L-Proline, 5.0-6.5 wt. % L-Serine, 0.4-0.6 wt. % Taurine, 5-6 wt. % L-Threonine, 1.9-2.2 wt. % L-Tryptophan, 0.5-1.0 wt. % L-Tyrosine, 7-8 wt. % L-Valine, 5.5-7.0 wt. % aspartic acid, and 10.0-11.7 wt. % glutamic acid, based on the total weight of the amino acids, and in the third chamber 17.4-18.4 ml of a lipid emulsion providing 20-22 g of lipids per 100 ml of the lipid emulsion and comprising 14-19 wt. % caprylic acid, 10-14 wt. % capric acid, 24-29 wt. % oleic acid, 16-21 wt. % linoleic acid, 1.5-3.5 wt. % eicosapentaenoic acid and 2-3 wt. % docosahexaenoic acid based on the total weight of the lipids, wherein the fatty acids are present in triglyceride-bound form, preferably wherein the lipid emulsion comprises soybean oil, medium-chain triglycerides, olive oil and fish oil, more preferably wherein the lipid emulsion comprises 30 wt. % soybean oil, 30 wt. % medium-chain triglycerides, 25 wt. % olive oil and 15 wt. % fish oil based on the total weight of the lipids.

In another preferred embodiment, the 3-chamber bag comprises in the first chamber 254 to 256 ml of a glucose solution comprising 19.5 to 19.7 g of glucose per 100 ml of the glucose solution, in the second chamber 191 to 192 ml of an amino acid solution providing 6.3 to 6.7 g of amino acids per 100 ml of the amino acid solution and comprising 8-10 wt. % L-Alanine, 6-9 wt. % L-Arginine, 0.5-2 wt. % L-Cysteine and/or L-Cystine, 3-5 wt. % Glycine, 3-5 wt. % L-Histidine, 4-8 wt. % L-Isoleucine, 10-13 wt. % L-Leucine, 8-11 wt. % L-Lysine, 2-4 wt. % L-Methionine, 3-5 wt. % L-Phenylalanine, 3-10 wt. % L-Proline, 4-8 wt. % L-Serine, 0.3-0.7 wt. % Taurine, 3-6 wt. % L-Threonine, 1.8-2.2 wt. %

L-Tryptophan, 0.4-4.2 wt. % L-Tyrosine, and 5-9 wt. % L-Valine, based on the total weight of the amino acids or of an amino acid solution providing 6.3 to 6.7 g of amino acids per 100 ml of the amino acid solution and comprising 8.0-9.7 wt. % L-alanine, 6.2-8.4 wt. % L-arginine, 0.5-1.9 wt. %-Cysteine and/or L-Cystine, 3-5 wt. % Glycine, 3.2-4.8 wt. % L-Histidine, 4.7-8.0 wt. % L-Isoleucine, 10.0-13.0 wt. % L-Leucine, 8.5-11.0 wt. % L-Lysine, 1.9-3.2 wt. % L-Methionine, 3.7-4.2 wt. % L-Phenylalanine, 3.0-9.7 wt. % L-Proline, 4.0-7.7 wt. % L-Serine, 0.4-0.6 wt. % Taurine, 3.7-5.5 wt. % L-Threonine, 2.0-2.2 wt. % L-Tryptophan, 0.4-4.2 wt. % L-Tyrosine, and 5.5-9.0 wt. % L-Valine, based on the total weight of the amino acids, preferably of an amino acid solution providing 6.4 to 6.7 g amino acids per 100 ml of the amino acid solution and comprising 9-10 wt. % L-Alanine, 5.5-7.0 wt. % L-Arginine, 1-2 wt. % L-Cysteine and/or L-Cystine, 2.5-4.0 wt. % Glycine, 2.5-4.0 wt. % L-Histidine, 4.0-5.5 wt. % L-Isoleucine, 10.0-11.5 wt. % L-Leucine, 8-9 wt. % L-Lysine, 1.5-2.5 wt. % L-Methionine, 3.5-5.0 wt. % L-Phenylalanine, 8-9 wt. % L-Proline, 5.0-6.5 wt. % L-Serine, 0.4-0.6 wt. % Taurine, 5-6 wt. % L-Threonine, 1.9-2.2 wt. % L-Tryptophan, 0.5-1.0 wt. % L-Tyrosine, 7-8 wt. % L-Valine, 5.5-7.0 wt. % aspartic acid, and 10.0-11.7 wt. % glutamic acid, based on the total weight of the amino acids, the amino acid solution preferably further comprising Ca²⁺, Mg²⁺, Na⁺, K⁺, Cl⁻, and phosphate, and in the third chamber 53 to 54 ml of a lipid emulsion providing 20 to 22 g of lipids per 100 ml of the lipid emulsion and comprising 14-19 wt. % caprylic acid, 10-14 wt. % capric acid, 24-29 wt. % oleic acid, 16-21 wt. % linoleic acid, 1.5-3.5 wt. % eicosapentaenoic acid and 2-3 wt. % docosahexaenoic acid based on the total weight of the lipids, wherein the fatty acids are present in triglyceride-bound form, preferably wherein the lipid emulsion comprises soybean oil, medium-chain triglycerides, olive oil and fish oil, more preferably wherein the lipid emulsion comprises 30 wt. % soybean oil, 30 wt. % medium-chain triglycerides, 25 wt. % olive oil and 15 wt. % fish oil based on the total weight of the lipids.

In yet another preferred embodiment, the 3-chamber bag comprises in the first chamber 571 to 574 ml of a glucose solution comprising 17.5 to 18.4 g of glucose per 100 ml of the glucose solution, in the second chamber 318 to 320 ml of an amino acid solution providing 6.3 to 6.7 g of amino acids per 100 ml of the amino acid solution and comprising 8-10 wt. % L-Alanine, 6-9 wt. % L-Arginine, 0.5-2 wt. % L-Cysteine and/or L-Cystine, 3-5 wt. % Glycine, 3-5 wt. % L-Histidine, 4-8 wt. % L-Isoleucine, 10-13 wt. % L-Leucine, 8-11 wt. % L-Lysine, 2-4 wt. % L-Methionine, 3-5 wt. % L-Phenylalanine, 3-10 wt. % L-Proline, 4-8 wt. % L-Serine, 0.3-0.7 wt. % Taurine, 3-6 wt. % L-Threonine, 1.8-2.2 wt. % L-Tryptophan, 0.4-4.2 wt. % L-Tyrosine, and 5-9 wt. % L-Valine, based on the total weight of the amino acids or of an amino acid solution providing 6.3 to 6.7 g of amino acids per 100 ml of the amino acid solution and comprising 8.0-9.7 wt. % L-alanine, 6.2-8.4 wt. % L-arginine, 0.5-1.9 wt. %-Cysteine and/or L-Cystine, 3-5 wt. % Glycine, 3.2-4.8 wt. % L-Histidine, 4.7-8.0 wt. % L-Isoleucine, 10.0-13.0 wt. % L-Leucine, 8.5-11.0 wt. % L-Lysine, 1.9-3.2 wt. %

L-Methionine, 3.7-4.2 wt. % L-Phenylalanine, 3.0-9.7 wt. % L-Proline, 4.0-7.7 wt. % L-Serine, 0.4-0.6 wt. % Taurine, 3.7-5.5 wt. % L-Threonine, 2.0-2.2 wt. % L-Tryptophan, 0.4-4.2 wt. % L-Tyrosine, and 5.5-9.0 wt. % L-Valine, based on the total weight of the amino acids, preferably of an amino acid solution providing 6.4 to 6.7 g amino acids per 100 ml of the amino acid solution and comprising 9-10 wt. % L-Alanine, 5.5-7.0 wt. % L-Arginine, 1-2 wt. % L-Cysteine and/or L-Cystine, 2.5-4.0 wt. % Glycine, 2.5-4.0 wt. % L-Histidine, 4.0-5.5 wt. % L-Isoleucine, 10.0-11.5 wt. % L-Leucine, 8-9 wt. % L-Lysine, 1.5-2.5 wt. % L-Methionine, 3.5-5.0 wt. % L-Phenylalanine, 8-9 wt. % L-Proline, 5.0-6.5 wt. % L-Serine, 0.4-0.6 wt. % Taurine, 5-6 wt. % L-Threonine, 1.9-2.2 wt. % L-Tryptophan, 0.5-1.0 wt. % L-Tyrosine, 7-8 wt. % L-Valine, 5.5-7.0 wt. % aspartic acid, and 10.0-11.7 wt. % glutamic acid, based on the total weight of the amino acids, the amino acid solution preferably further comprising Ca²⁺, Mg²⁺, Na⁺, K⁺, Cl⁻, and phosphate, and in the third chamber 108 to 109 ml of a lipid emulsion providing 20 to 22 g of lipids per 100ml of the lipid emulsion and comprising 14-19 wt. % caprylic acid, 10-14 wt. % capric acid, 24-29 wt. % oleic acid, 16-21 wt. % linoleic acid, 1.5-3.5 wt. % eicosapentaenoic acid and 2-3 wt. % docosahexaenoic acid based on the total weight of the lipids, wherein the fatty acids are present in triglyceride-bound form, preferably wherein the lipid emulsion comprises soybean oil, medium-chain triglycerides, olive oil and fish oil, more preferably wherein the lipid emulsion comprises 30 wt. % soybean oil, 30 wt. % medium-chain triglycerides, 25 wt. % olive oil and 15 wt. % fish oil based on the total weight of the lipids.

In a further preferred embodiment, the 3-chamber bag comprises in the first chamber 857 to 861 ml of a glucose solution comprising 17.5 to 18.4 g of glucose per 100 ml of the glucose solution, in the second chamber 477 to 480 ml of an amino acid solution providing 6.3 to 6.7 g of amino acids per 100 ml of the amino acid solution and comprising 8-10 wt. % L-Alanine, 6-9 wt. % L-Arginine, 0.5-2 wt. % L-Cysteine and/or L-Cystine, 3-5 wt. % Glycine, 3-5 wt. % L-Histidine, 4-8 wt. % L-Isoleucine, 10-13 wt. % L-Leucine, 8-11 wt. % L-Lysine, 2-4 wt. % L-Methionine, 3-5 wt. % L-Phenylalanine, 3-10 wt. % L-Proline, 4-8 wt. % L-Serine, 0.3-0.7 wt. % Taurine, 3-6 wt. % L-Threonine, 1.8-2.2 wt. % L-Tryptophan, 0.4-4.2 wt. % L-Tyrosine, and 5-9 wt. % L-Valine, based on the total weight of the amino acids or of an amino acid solution providing 6.3 to 6.7 g of amino acids per 100 ml of the amino acid solution and comprising 8.0-9.7 wt. % L-alanine, 6.2-8.4 wt. % L-arginine, 0.5-1.9 wt. %-Cysteine and/or L-Cystine, 3-5 wt. % Glycine, 3.2-4.8 wt. % L-Histidine, 4.7-8.0 wt. % L-Isoleucine, 10.0-13.0 wt. % L-Leucine, 8.5-11.0 wt. % L-Lysine, 1.9-3.2 wt. % L-Methionine, 3.7-4.2 wt. % L-Phenylalanine, 3.0-9.7 wt. % L-Proline, 4.0-7.7 wt. % L-Serine, 0.4-0.6 wt. % Taurine, 3.7-5.5 wt. % L-Threonine, 2.0-2.2 wt. % L-Tryptophan, 0.4-4.2 wt. % L-Tyrosine, and 5.5-9.0 wt. % L-Valine, based on the total weight of the amino acids, preferably of an amino acid solution providing 6.4 to 6.7 g amino acids per 100 ml of the amino acid solution and comprising 9-10 wt. % L-Alanine, 5.5-7.0 wt. % L-Arginine, 1-2 wt. % L-Cysteine and/or L-Cystine, 2.5-4.0 wt. % Glycine, 2.5-4.0 wt. % L-Histidine, 4.0-5.5 wt. % L-Isoleucine, 10.0-11.5 wt. % L-Leucine, 8-9 wt. % L-Lysine, 1.5-2.5 wt. % L-Methionine, 3.5-5.0 wt. % L-Phenylalanine, 8-9 wt. % L-Proline, 5.0-6.5 wt. % L-Serine, 0.4-0.6 wt. % Taurine, 5-6 wt. % L-Threonine, 1.9-2.2 wt. % L-Tryptophan, 0.5-1.0 wt. % L-Tyrosine, 7-8 wt. % L-Valine, 5.5-7.0 wt. % aspartic acid, and 10.0-11.7 wt. % glutamic acid, based on the total weight of the amino acids, the amino acid solution preferably further comprising Ca^{2+,} Mg^{2+,} Na⁺, K³⁰ , Cl⁻, and phosphate, and in the third chamber 162 to 163 ml of a lipid emulsion providing 20 to 22 g of lipids per 100 ml of the lipid emulsion and comprising 14-19 wt. % caprylic acid, 10-14 wt. % capric acid, 24-29 wt. % oleic acid, 16-21 wt. % linoleic acid, 1.5-3.5 wt. % eicosapentaenoic acid and 2-3 wt. % docosahexaenoic acid based on the total weight of the lipids, wherein the fatty acids are present in triglyceride-bound form, preferably wherein the lipid emulsion comprises soybean oil, medium-chain triglycerides, olive oil and fish oil, more preferably wherein the lipid emulsion comprises 30 wt. % soybean oil, 30 wt. % medium-chain triglycerides, 25 wt. % olive oil and 15 wt. % fish oil based on the total weight of the lipids.

In a particularly preferred embodiment, the present invention relates to a composition for use in providing parenteral nutrition to a pediatric patient, wherein the composition is comprised in a 3-chamber bag comprising 18 ml of the lipid emulsion according to example 1 below, 108 ml of the amino acid solution according to example 2a below and 124 ml of a glucose solution comprising 21.6 g glucose per 100 ml of the glucose solution, wherein the contents of the 3 chambers is mixed before the composition is intravenously administered to the pediatric patient, wherein the pediatric patient is an infant, preferably a preterm infant, and wherein the composition is administered at a dose of 70-80 ml per kg body weight per day on day 1 after birth and at a dose of 90-100 ml per kg body weight per day on day 2 after birth.

In further particularly preferred embodiment the present invention relates to a composition for use in providing parenteral nutrition to a pediatric patient, wherein the composition is comprised in a 3-chamber bag comprising 54 ml of the lipid emulsion according to example 1 below, 191 ml of the amino acid solution according to example 2b below and 255 ml of a glucose solution comprising 19.6 g glucose per 100 ml of the glucose solution, wherein the contents of the 3 chambers is mixed before the composition is intravenously administered to the pediatric patient, wherein the pediatric patient is an infant, preferably a preterm infant, and wherein the composition is administered at a dose of 120-140 ml per kg body weight per day from day 3 to day 28 after birth.

In another particularly preferred embodiment the present invention relates to a composition for use in providing parenteral nutrition to a pediatric patient, wherein the composition is comprised in a 3-chamber bag comprising 108 ml of the lipid emulsion according to example 1 below, 319 ml of the amino acid solution according to example 2c below and 573 ml of a glucose solution comprising 18.2 g glucose per 100 ml of the glucose solution, wherein the contents of the 3 chambers is mixed before the composition is intravenously administered to the pediatric patient, wherein where the pediatric patient is a newborn infant between 1 day and 27 days of age, the composition is administered at a dose of 110-130 ml, preferably 120 ml, per kg body weight per day, where the pediatric patient is between 27 days and 2 years of age the composition is administered at a dose of 80-110 ml, preferably 90-100 ml, per kg body weight per day, where the pediatric patient is between 2 and 11 years of age the composition is administered at a dose of 50-90 ml, preferably 60-80 ml, per kg body weight per day, and where the pediatric patient is between 12 and 18 years of age the composition is administered at a dose of 30-60 ml, preferably 40-50 ml per kg body weight per day.

In yet another particularly preferred embodiment the present invention relates to a composition for use in providing parenteral nutrition to a pediatric patient, wherein the composition is comprised in a 3-chamber bag comprising 162.5 ml of the lipid emulsion according to example 1 below, 478.5 ml of the amino acid solution according to example 2c below and 859 ml of a glucose solution comprising 18.2 g glucose per 100 ml of the glucose solution, wherein the contents of the 3 chambers is mixed before the composition is intravenously administered to the pediatric patient, wherein where the pediatric patient is a newborn infant between 1 day and 27 days of age, the composition is administered at a dose of 110-130 ml, preferably 120 ml, per kg body weight per day, where the pediatric patient is between 27 days and 2 years of age the composition is administered at a dose of 80-110 ml, preferably 90-100 ml, per kg body weight per day, where the pediatric patient is between 2 and 11 years of age the composition is administered at a dose of 50-90 ml, preferably 60-80 ml, per kg body weight per day, and where the pediatric patient is between 12 and 18 years of age the composition is administered at a dose of 30-60 ml, preferably 40-50 ml per kg body weight per day.

### EXAMPLES

### Example 1

The emulsion was prepared from the ingredients listed in table 1.

TABLE 1

| ingredient | amount (g) |
|---|---|
| glycerol | 50.00 |
| emulsifier (either PL1 or PL2) | 24.00 |
| sodium oleate | 0.60 |
| NaOH 1M | q.s. |
| Soybean oil | 120.00 |
| MCT oil | 120.00 |
| olive oil | 100.00 |
| fish oil | 60.00 |
| alpha tocopherol | 0.04 |
| WFI | ad 2000 |
| nitrogen | q.s. |

The lipid emulsion was prepared according to the following process:

Glycerol, sodium oleate and 325 ml of water for injection were mixed and heated to 60 to 70° C. Then, the emulsifier was added under stirring by means of an Ultra-Turrax (T50) at 5000 rpm for 5 minutes to obtain the water phase 1.

The pH of the water phase 1 was adjusted to 9.0 to 10.0 by adding a solution of sodium hydroxide.

The oil phase was provided by mixing the four oils and alpha tocopherol. The oil phase was heated to 60 to 70° C.

The oil phase and the water phase 1 were mixed at 60 to 70° C. by means of an Ultra-Turrax (T50) for 10 to 12 minutes at 10000 rpm. The pH was adjusted to 9.0 to 10.0 by adding a solution of sodium hydroxide.

The pre-emulsion was then homogenized in a high-pressure valve homogenizer in 6 cycles at a pressure of 560 bar in the first stage and at a pressure of 120 bar in the second stage (APV-1000, SPX Flow Technology).

The residual water for injection (=water phase 2) was added to adjust the volume of the emulsion to 2 liters, and the pH was adjusted to 9.0 to 10.0.

### Example 2a

An amino acid solution comprising 65.3 g amino acids per liter was prepared by dissolving 6.3 g L-alanine, 4.1 g L-arginine, 4.1 g L-aspartic acid, 1.0 g L-cysteine/L-cystine, 7.1g L-glutamic acid, 2.1g glycine, 2.1g L-histidine, 3.1g L-isoleucine, 7.0 g L-leucine, 5.6 g L-lysine, 1.3 g L-methionine, 2.7 g L-phenylalanine, 5.6 g L-proline, 3.8 g L-serine, 0.3 g taurine, 3.6 g L-threonine, 1.4 g L-tryptophan, 0.5 g L-tyrosine, and 3.6 g L-valine in water for injection by stirring until the amino acids had completely dissolved and then adjusting the volume to one liter.

The pH of the solution was adjusted to 5.2 by adding a solution of acetic acid, the osmolarity was 520 mOsmol/kg.

### Example 2b

An amino acid solution comprising 65.3 g amino acids per liter was prepared by dissolving 6.3 g L-alanine, 4.1 g L-arginine, 4.1 g L-aspartic acid, 1.0 g L-cysteine/L-cystine, 7.1 g L-glutamic acid, 2.1 g glycine, 2.1 g L-histidine, 3.1 g L-isoleucine, 7.0 g L-leucine, 5.6 g L-lysine, 1.3 g L-methionine, 2.7 g L-phenylalanine, 5.6 g L-proline, 3.8 g L-serine, 0.3 g taurine, 3.6 g L-threonine, 1.4 g L-tryptophan, 0.5 g L-tyrosine, and 3.6 g L-valine as well as 30 mMol Ca²⁺(as calcium gluconate monohydrate), 4 mmol Mg²⁺(as magnesium sulfate heptahydrate), 52 mMol Na⁺(as sodium glycerophosphate), 47 mMol K⁺(as potassium chloride), 47 mMol Cl⁻(as potassium chloride), 26 mMol phosphate (as sodium glycerophosphate), and 4 mmol sulfate (as magnesium sulfate heptahydrate) in water for injection by stirring until the solids had completely dissolved and then adjusting the volume to one liter.

The pH of the solution was adjusted to 5.2.

The osmolarity of the solution was 880 mOsmol/kg.

### Example 2c

An amino acid solution comprising 65.3 g amino acids per liter was prepared by dissolving 6.3 g L-alanine, 4.1 g L-arginine, 4.1 g L-aspartic acid, 1.0 g L-cysteine/L-cystine, 7.1 g L-glutamic acid, 2.1 g glycine, 2.1 g L-histidine, 3.1 g L-isoleucine, 7.0 g L-leucine, 5.6 g L-lysine, 1.3 g L-methionine, 2.7 g L-phenylalanine, 5.6 g L-proline, 3.8 g L-serine, 0.3 g taurine, 3.6 g L-threonine, 1.4 g L-tryptophan, 0.5 g L-tyrosine, and 3.6 g L-valine as well as 21 mMol Ca²⁺(as calcium gluconate monohydrate), 5 mMol Mg²⁺(as magnesium sulfate heptahydrate), 57 mMol Na⁺(as sodium acetate trihydrate and sodium glycerophosphate), 52 mMol K⁺(as potassium chloride), 52 mMol Cl⁻(as potassium chloride), 15 mMol acetate (as sodium acetate trihydrate), 21 mMol phosphate (as sodium glycerophosphate), and 5 mMol sulfate (as magnesium sulfate heptahydrate) in water for injection by stirring until the solids had completely dissolved and then adjusting the volume to one liter.

The pH of the solution was adjusted to 5.2.

The osmolarity of the solution was 880 mOsmol/kg.

### Example 3

A 3-chamber-bag was filled to contain 18 ml of the lipid emulsion according to example 1, 108 ml of the amino acid solution according to example 2a and 124 ml of a glucose solution comprising 21.6 g glucose per 100 ml of the glucose solution.

The bag was autoclaved at 121.1° C. for 15 minutes.

### Example 4

A 3-chamber-bag was filled to contain 54 ml of the lipid emulsion according to example 1, 191 ml of the amino acid solution according to example 2b and 255 ml of a glucose solution comprising 19.6 g glucose per 100 ml of the glucose solution.

The bag was autoclaved at 121.1° C. for 15 minutes.

### Example 5

A 3-chamber-bag was filled to contain 108 ml of the lipid emulsion according to example 1, 319 ml of the amino acid solution according to example 2c and 573 ml of a glucose solution comprising 18.2 g glucose per 100 ml of the glucose solution.

The bag was autoclaved at 121.1° C. for 15 minutes.

### Example 6

A 3-chamber-bag was filled to contain 162.5 ml of the lipid emulsion according to example 1, 478.5 ml of the amino acid solution according to example 2c and 859 ml of a glucose solution comprising 18.2 g glucose per 100 ml of the glucose solution.

The bag was autoclaved at 121.1° C. for 15 minutes.

1. A composition for use in providing parenteral nutrition to a pediatric patient wherein the composition is comprised in a 3-chamber bag and comprises:
(i) in the first chamber a glucose solution providing 14-22 g glucose per 100 ml of the glucose solution,
(ii) in the second chamber an amino acid solution providing 6.3-6.7 g of amino acids per 100 ml of the amino acid solution and comprising 8-10 wt. % L-Alanine, 6-9 wt. % L-Arginine, 0.5-2 wt. % L-Cysteine and/or L-Cystine, 3-5 wt. % Glycine, 3-5 wt. % L-Histidine, 4-8 wt. % L-Isoleucine, 10-13 wt. % L-Leucine, 8-11 wt. % L-Lysine, 2-4 wt. % L-Methionine, 3-5 wt. % L-Phenylalanine, 3-10 wt. % L-Proline, 4-8 wt. % L-Serine, 0.3-0.7 wt. % Taurine, 3-6 wt. % L-Threonine, 1.8-2.2 wt. % L-Tryptophan, 0.4-4.2 wt. % L-Tyrosine, and 5-9 wt. % L-Valine, based on the total weight of the amino acids, and
(iii) in the third chamber a lipid emulsion providing 20-22 g of lipids per 100 ml of the lipid emulsion and comprising 14-19 wt. % caprylic acid, 10-14 wt. % capric acid, 24-29 wt. % oleic acid, 16-21 wt. % linoleic acid, 1.5-3.5 wt. % eicosapentaenoic acid and 2-3 wt. % docosahexaenoic acid based on the total weight of the lipids, wherein the fatty acids are present in triglyceride-bound form,
wherein the contents of the 3 chambers is mixed before it is intravenously administered to the pediatric patient, wherein the glucose solution, the amino acid solution and the lipid emulsion are comprised in the composition at a volume ratio of 4.6-7.1:2.9-6.1:1, and wherein the composition is administered at a dose of 30-140 ml per kg body weight per day.

2. The composition according to claim 1, wherein the amino acid solution comprises 8.0-9.7 wt. % L-alanine, 6.2-8.4 wt. % L-arginine, 0.5-1.9 wt. %-Cysteine and/or L-Cystine, 3-5 wt. % Glycine, 3.2-4.8 wt. % L-Histidine, 4.7-8.0 wt. % L-Isoleucine, 10.0-13.0 wt. % L-Leucine, 8.5-11.0 wt. % L-Lysine, 1.9-3.2 wt. % L-Methionine, 3.7-4.2 wt. % L-Phenylalanine, 3.0-9.7 wt. % L-Proline, 4.0-7.7 wt. % L-Serine, 0.4-0.6 wt. % Taurine, 3.7-5.5 wt. % L-32 Threonine, 2.0-2.2 wt. % L-Tryptophan, 0.4-4.2 wt. % L-Tyrosine, and 5.5-9.0 wt. % L-Valine, based on the total weight of the amino acids.

3. The composition according to claim 1, wherein the amino acid solution provides 6.4-6.7 g amino acids per 100 ml of the amino acid solution and comprises 9-10 wt. % L-Alanine, 5.5-7.0 wt. % L-Arginine, 1-2 wt. % L-Cysteine and/or L-Cystine, 2.5-4.0 wt. % Glycine, 2.5-4.0 wt. % L-Histidine, 4.0-5.5 wt. % L-Isoleucine, 10.0-11.5 wt. % L-Leucine, 8-9 wt. % L-Lysine, 1.5-2.5 wt. % L-Methionine, 3.5-5.0 wt. % L-Phenylalanine, 8-9 wt. % L-Proline, 5.0-6.5 wt. % L-Serine, 0.4-0.6 wt. % Taurine, 5-6 wt. % L-Threonine, 1.9-2.2 wt. % L-Tryptophan, 0.5-1.0 wt. % L-Tyrosine, 7-8 wt. % L-Valine, 5.5-7.0 wt. % aspartic acid, and 10.0-11.7 wt. % glutamic acid, based on the total weight of the amino acids.

4. The composition according to claim 1, wherein the lipid emulsion comprises soybean oil, medium-chain triglycerides, olive oil and fish oil, preferably wherein the lipid emulsion comprises 30 wt. % soybean oil, 30 wt. % medium-chain triglycerides, 25 wt. % olive oil and 15 wt. % fish oil based on the total weight of the lipids.

5. The composition according to claim 1, wherein the composition is comprised in a 3-chamber bag comprising:
(i) in the first chamber a glucose solution providing 21-22 g, preferably 21.6 g, glucose per 100 ml of the glucose solution,
(ii) in the second chamber an amino acid solution according to claim 1, and
(iii) in the third chamber a lipid emulsion according to claim 1, wherein the contents of the 3 chambers is mixed before the composition is intravenously administered to the pediatric patient, preferably wherein after mixing the contents of the 3 chambers the composition has an osmolarity of 830-850 mOsmol/kg, preferably 840 mOsmol/kg, wherein the glucose solution, the amino acid solution and the lipid emulsion are administered at a volume ratio of 6.8-7.0:5.9-6.1:1, preferably at a volume ratio of 6.9:6.0:1,
wherein the pediatric patient is an infant, preferably a preterm infant, and wherein the composition is administered at a dose of 70-80 ml per kg body weight per day on day 1 after birth and at a dose of 90-100 ml per kg body weight per day on day 2 after birth.

6. The composition according to claim 5, wherein the first chamber comprises 123-125 ml of the glucose solution, wherein the second chamber comprises 107-109 ml of the amino acid solution, and wherein the third chamber comprises 17.4-18.4 ml of the lipid emulsion.

7. The composition according to claim 5, comprising
(i) in the first chamber 123-125 ml of a glucose solution providing 21.6 g glucose per 100 ml of the glucose solution,
(ii) in the second chamber 107-109 ml of the amino acid solution according to claim 3, and
(iii) in the third chamber 17.4-18.4 ml of the lipid emulsion according to claim 4 comprising 30 wt. % soybean oil, 30 wt. % medium-chain triglycerides, 25 wt. % olive oil and 15 wt. % fish oil based on the total weight of the lipids, wherein the contents of the 3 chambers is mixed before the composition is intravenously administered to the pediatric patient and wherein after mixing the contents of the 3 chambers the composition has an osmolarity 840 mOsmol/kg, wherein the glucose solution, the amino acid solution and the lipid emulsion are administered at a volume ratio of 6.9:6.0:1, and wherein the pediatric patient is a preterm infant.

8. The composition according to claim 1, wherein the composition is comprised in a 3-chamber bag comprising:
(i) in the first chamber a glucose solution providing 19.5-19.7 g, preferably 19.6 g, glucose per 100 ml of the glucose solution,
(ii) in the second chamber an amino acid solution according to any of claims 1 to 3, optionally further comprising Ca²⁺, Mg²⁺, Na⁺, K⁺, Cl⁻, and phosphate, and
(iii) in the third chamber a lipid emulsion according to claim 1, wherein the contents of the 3 chambers is mixed before the composition is intravenously administered to the pediatric patient, preferably wherein after mixing the contents of the 3 chambers the composition has an osmolarity of 880-920 mOsmol/kg, wherein the glucose solution, the amino acid solution and the lipid emulsion are administered at a volume ratio of 4.5-4.7:3.4-3.6:1, preferably at a volume ratio of 4.6:3.5:1, wherein the pediatric patient is an infant, preferably a preterm infant, and wherein the composition is administered at a dose of 120-140 ml per kg body weight per day from day 3 to day 28 after birth.

9. The composition according to claim 8, wherein the first chamber comprises 254-256 ml of the glucose solution, wherein the second chamber comprises 191-192 ml of the amino acid solution, and wherein the third chamber comprises 53-54 ml of the lipid emulsion.

10. The composition according to claim 8, comprising:
(i) in the first chamber 254-256 ml of a glucose solution providing 19.6 g, glucose per 100 ml of the glucose solution,
(ii) in the second chamber 191-192 ml of the amino acid solution according to claim 3, further comprising Ca²⁺, Mg²⁺, Na⁺, K⁺, Cl⁻, and phosphate, and
(iii) in the third chamber 53-54 ml of the lipid emulsion according to claim 4, wherein the glucose solution, the amino acid solution and the lipid emulsion are administered at a volume ratio of 4.7:3.5:1, and wherein the pediatric patient is a preterm infant.

11. The composition according to claim 1, wherein the composition is comprised in a 3-chamber bag comprising
(i) in the first chamber a glucose solution providing 17.5-18.4 g, preferably 18.2 g, glucose per 100 ml of the glucose solution,
(ii) in the second chamber an amino acid, optionally further comprising Ca²⁺, Mg²⁺, Na⁺, K⁺, Cl⁻, and phosphate, and (Hi) in the third chamber a lipid emulsion according to claim 1, wherein the contents of the 3 chambers is mixed before the composition is intravenously administered to the pediatric patient, preferably wherein after mixing the contents of the 3 chambers the composition has an osmolarity of 850-870 or 880-900 mOsmol/kg, wherein the glucose solution, the amino acid solution and the lipid emulsion are administered at a volume ratio of 5.2-5.4:2.9-3.1:1, preferably at a volume ratio of 5.3:3.0:1, and wherein, where the pediatric patient is a newborn infant between 1 day and 27 days of age, the composition is administered at a dose of 110-130 ml, preferably 120 ml, per kg body weight per day, where the pediatric patient is between 27 days and 2 years of age the composition is administered at a dose of 80-110 ml, preferably 90-100 ml, per kg body weight per day, where the pediatric patient is between 2 and 11 years of age the composition is administered at a dose of 50-90 ml, preferably 60-80 ml, per kg body weight per day, and where the pediatric patient is between 12 and 18 years of age the composition is administered at a dose of 30-60 ml, preferably 40-50 ml per kg body weight per day.

12. The composition according to claim 11, wherein the first chamber comprises 571-574 ml of the glucose solution, wherein the second chamber comprises 318-320 ml of the amino acid solution, and wherein the third chamber comprises 108-109 ml of the lipid emulsion.

13. The composition according to claim 12, comprising:
(i) in the first chamber 571-574 ml of a glucose solution providing 18.2 g, glucose per 100 ml of the glucose solution,
(ii) in the second chamber 318-320 ml of the amino acid according to claim 3, further comprising Ca²⁺, Mg²⁺, Na⁺, K⁺, Cl⁻, and phosphate, and
(iii) in the third chamber 108-109 ml of the lipid emulsion according to claim 4, wherein the glucose solution, the amino acid solution and the lipid emulsion are administered at a volume ratio of 5.3:3.0:1, and wherein where the pediatric patient is a newborn infant between 1 day and 27 days of age, the composition is administered at a dose of 120 ml, where the pediatric patient is between 27 days and 2 years of age the composition is administered at a dose of 90-100 ml, per kg body weight per day, where the pediatric patient is between 2 and 11 years of age the composition is administered at a dose of 60-80 ml, per kg body weight per day, and where the pediatric patient is between 12 and 18 years of age the composition is administered at a dose of 40-50 ml per kg body weight per day.

14. The composition according to claim 11, wherein the first chamber comprises 857-861 ml of the glucose solution, wherein the second chamber comprises 477-480 ml of the amino acid solution, and wherein the third chamber comprises 162-163 ml of the lipid emulsion.

15. The composition according to claim 14 comprising:
(i) in the first chamber 857-861 ml of a glucose solution providing 18.2 g, glucose per 100 ml of the glucose solution,
(ii) in the second chamber 477-480 ml of the amino acid according to claim 3, further comprising Ca²⁺, Mg²⁺, Na⁺, K⁺, phosphate and Cl⁻,
(iii) in the third chamber 162-163 ml of the lipid emulsion according to claim 4, wherein the glucose solution, the amino acid solution and the lipid emulsion are administered at a volume ratio of 5.3:3.0:1, and wherein where the pediatric patient is a newborn infant the composition is administered at a dose of 120 ml, per kg body weight per day from day 1 to day 27 after birth, where the pediatric patient is between 27 days and 2 years of age the composition is administered at a dose of 90-100 ml, per kg body weight per day, where the pediatric patient is between 2 and 11 years of age the composition is administered at a dose of 60-80 ml, per kg body weight per day, and
where the pediatric patient is between 12 and 18 years of age the composition is administered at a dose of 40-50 ml per kg body weight per day.

16. A 3-chamber bag comprising in the first chamber a glucose solution providing 14-22 g glucose per 100 ml of the glucose solution in the second chamber an amino acid solution providing 6.3-6.7 g of amino acids per 100 ml of the amino acid solution and comprising 8-10 wt. % L-Alanine, 6-9 wt. % L-Arginine, 0.5-2 wt. % L-Cysteine and/or L-Cystine, 3-5 wt. % Glycine, 3-5 wt. % L-Histidine, 4-8 wt. % L-Isoleucine, 10-13 wt. % L-Leucine, 8-11 wt. % L-Lysine, 2-4 wt. % L-Methionine, 3-5 wt. % L-Phenylalanine, 3-10 wt. % L-Proline, 4-8 wt. % L-Serine, 0.3-0.7 wt. % Taurine, 3-6 wt. % L-Threonine, 1.8-2.2 wt. % L-Tryptophan, 0.4-4.2 wt. % L-Tyrosine, and 5-9 wt. % L-Valine, based on the total weight of the amino acids, and in the third chamber a lipid emulsion providing 20-22 g of lipids per 100 ml of the lipid emulsion and comprising 14-19 wt. % caprylic acid, 10-14 wt. % capric acid, 24-29 wt. % oleic acid, 16-21 wt. % linoleic acid, 1.5-3.5 wt. % eicosapentaenoic acid and 2-3 wt. % docosahexaenoic acid based on the total weight of the lipids, wherein the fatty acids are present in triglyceride-bound form.

17. The 3-chamber bag according to claim 16, comprising in the first chamber 123-125 ml of a glucose solution proving 21-22 g glucose per 100 ml of the glucose solution, in the second chamber 107-109 ml of the amino acid solution, and in the third chamber 17.4-18.4 ml of the lipid emulsion.

18. The 3-chamber bag according to claim 16, comprising in the first chamber 254-256 ml of a glucose solution comprising 19.5-19.7 g of glucose per 100 ml of the glucose solution, in the second chamber 191-192 ml of the amino acid solution, the amino acid solution preferably further comprising Ca²⁺, Mg²⁺, Nat, K⁺, Cl₋, and phosphate, and in the third chamber 53-54 ml of the lipid emulsion.

19. The 3-chamber bag according to claim 16, comprising in the first chamber 571-574 ml of a glucose solution comprising 17.5-18.4 g of glucose per 100 ml of the glucose solution, in the second chamber 318-320 ml of the amino acid solution the amino acid solution preferably further comprising Ca²⁺, Mg²⁺, Na⁺, K⁺, Cl⁻, and phosphate, and in the third chamber 108-109 ml of the lipid emulsion.

20. The 3-chamber bag according to claim 16, comprising in the first chamber 857-861 ml of a glucose solution comprising 17.5-18.4 g of glucose per 100 ml of the glucose solution, in the second chamber 477-480 ml of the amino acid solution, the amino acid solution preferably further comprising Ca²⁺, Mg²⁺, Na⁺, K⁺, Cl⁻, and phosphate, and in the third chamber 162-163 ml of the lipid emulsion.
